(19) United States
(12) Patent Application Publication
(10) Pub. No.: US 2009/0068154 A1
(43) Pub. Date: Mar. 12, 2009

(54) Cell Composition for Transplant

(75) Inventor: Minoru Ueda, Tokyo (JP)

Correspondence Address: BIRCH STEWART KOLASCH & BIRCH, PO BOX 747, FALLS CHURCH, VA, 22040-0747, US

(73) Assignee: THE UNIVERSITY OF TOKYO, Tokyo (JP)

(21) Appl. No.: 12/087,338

(22) PCT Filed: Dec. 26, 2006
(86) PCT No.: PCT/JP2006/325909

§ 371 Date: Jul. 2, 2008

(30) Foreign Application Priority Data
Jan. 4, 2006 (JP) 2006-000075

(51) Int. Cl. A61K 35/12; C12N 5/06

(52) U.S. Cl. 424/93.7; 435/325; 435/371; 435/383

(57) ABSTRACT

The present invention provides a cell composition for transplant comprising fibroblasts originating in buccal fat pad.

### TECHNICAL FIELD

The present invention relates to a cell composition for transplant comprising lipofibroblasts originating in buccal fat pad.

### BACKGROUND ART

Various biological materials are used to repair abnormalities or defects in skin tissue caused by injury or illness, congenital or acquired (caused by aging) wrinkles, and deformities associated with depressions.

Examples of biochemical materials include bovine collagen (atherocollagen) (Delustro, F., et al., J. Biomed. Mater. Res., 1986, 20, 109-20) and hyaluronic acid gel (Duranti, F., et al., Dermatol. Surg., 1998, 24, 1317-25) having decreased antigenicity as a result of removing the C terminal and N terminal peptide portions.

However, bovine collagen and hyaluronic acid gel are easily hydrolyzed in the body, and have the shortcoming of the effects thereof not being sustainable as a result of being rapidly eliminated from an administration site. In addition, in the case of using foreign materials, it is difficult to completely eliminate immunological effects.

In addition, a cell composition obtained by in vitro culturing of fibroblasts originating in skin isolated from a patient's own skin tissue is known as an example of a cell composition for transplant (Japanese Unexamined International Patent Publication No. H11-510069).

Moreover, a cell composition for transplant has recently been developed by the inventors of the present invention that is obtained by in vitro culturing of fibroblasts originating in human gingiva (Japanese Patent Application No. 2005-190374).

Skin fibroblasts merely function as fillers, and do not have adequate functions for actively improving the physiological status of skin tissue at an affected area or peripheral regions thereof, namely the physical status and/or nutritional status of skin tissue.

In addition, since fibroblasts originating in skin and fibroblasts originating in gingiva are require the collection of skin tissue or gingival tissue, they cause considerable suffering for the patient and not considered to have an adequately low level of invasiveness. Moreover, these cells also have the shortcoming of a slow growth rate.

### DISCLOSURE OF THE INVENTION

### Problems to be Solved by the Invention

An object of the present invention is to provide a cell composition for transplant comprising fibroblasts originating in buccal fat pad.

### Means for Solving the Problems

As a result of conducting extensive studies, the inventors of the present invention found that lipofibroblasts originating in buccal fat pad have a superior growth rate and have a function that actively improves the physical status and/or nutritional status of skin tissue, thereby leading to completion of the present invention.

Thus, the present invention relates to the following:

1. a cell composition for transplant comprising fibroblasts originating in buccal fat pad;
2. the cell composition for transplant described in 1 above, wherein the buccal fat pad is of human origin;
3. the cell composition for transplant described in 2 above, wherein the buccal fat pad is autologous;
4. the cell composition for transplant described in any of 1 to 3 above for improving skin tissue;
5. a method for preparing the cell composition for transplant described in any of 1 to 4 above, comprising the steps of:
(a) collecting cells from buccal fat pad;
(b) culturing the collected cells in vitro; and
(c) recovering fibroblasts from the culture; and,
6. a method for improving skin tissue comprising the step of:
(a) administering the cell composition for transplant described in 4 above to a subject.

### EFFECTS OF THE INVENTION

The cell composition for transplant of the present invention demonstrates the effects of filling in sites of deformities or defects in tissue, and improving the physiological status of those sites and peripheral tissue thereof.

### BRIEF DESCRIPTION OF THE DRAWINGS

FIG. 1 shows a comparison of the growth rates of fibroblasts originating in skin dermis and buccal fat. Fibroblasts originating in buccal fat were determined to have a growth rate three times that of skin dermis on the basis of this data.

FIG. 2 shows a comparison of the growth factor production capabilities of fibroblasts originating in skin dermis and buccal fat. VEGF refers to vascular endothelial growth factor, while KGF refers to keratinocyte growth factor. The protein concentration of the culture broth (in pg/ml) is plotted on the vertical axis, while culturing time (in hours) is plotted on the horizontal axis. (a) indicates buccal fat, (b) indicates abdominal fat, and (c) indicates skin dermis.

### BEST MODE FOR CARRYING OUT THE INVENTION

Buccal fat is a fatty mass located in the depression below the zygomatic arch. The buccal fat pad is composed of a main portion and four branches (buccal branch, pterygoid branch, pterygomandibular branch and temporal branch). The buccal branch is located in the surface layer in the buccal portion, while the other three branches are located in deeper layers centering about the main portion. The main portion is located above the parotid duct, and extends along the upper portion of the anterior border of the masseter muscle.

The buccal fat pad of the present invention is from a mammal and preferably from a human. In addition, the buccal fat pad is also preferably autologous to avoid an immune rejection response.

The fibroblasts in the present invention refer to adhesive cells exhibiting a spindle shape during in vitro culturing and having the ability to produce collagen. In addition, the fibroblasts of the present invention preferably have the ability to produce various cell growth factors such as vascular endothelial growth factor (VEGF) and keratinocyte growth factor (KGF).

The fibroblasts of the present invention are preferably able to produce type I collagen. In addition, the ability to produce vascular endothelial growth factor of these fibroblasts is 40 to 60 pg/ml when treated for 24 hours, 55 to 75 pg/ml when treated for 48 hours and 75 to 80 pg/ml when treated for 72 hours, while the ability to produce keratinocyte growth factor is 1.3 to 1.8 ng/ml when treated for 24 hours, 1.8 to 2.4 ng/ml when treated for 48 hours, and 2.5 to 3.0 ng/ml when treated for 72 hours.

The fibroblasts of the present invention can include primary cultured cells and derived cells provided they retain the ability to produce collagen and cell growth factors. In addition, morphology and other characteristics may also differ provided these properties are shared.

The fibroblasts of the present invention can be collected by any conventionally known method such as centesis.

The collected cells can be cultured by any conventionally known method. In addition, a cell line can be established depending on the case.

A conventionally known culture broth used to culture fibroblasts can be used for the culture broth, examples of which include α-MEM medium, Eagle's medium and Dulbecco's modified Eagle's medium. An appropriate serum, antibiotics and the like can be suitably added to the culture broth.

The collected cells can be cultured to confluency in a suitable culture broth (37° C., 5% CO₂). The cultured cells can be subcultured as necessary. Although there are no restrictions on the number of rounds of subculturing provided the aforementioned properties are not lost, the number of rounds of subculturing is preferably 10 or less and more preferably 3 to 6.

Any vessel used to culture fibroblasts can be used for the culture vessel. Examples of culture vessel include a culture flask, culture dish or multiplate.

Fibroblasts are recovered from the culture vessel after having reached a desired number of cells. A conventional method used to recover fibroblasts can be used for the cell recovery method, and special procedures or treatment are not required to carry out the present invention. For example, cells adhered to the inner walls of a culture vessel can be treated with a suitable enzyme such as trypsin to separate the cells from the inner walls of the culture vessel and become isolated in the culture broth followed by recovery of the free cells together with the culture broth.

The recovered cells are cultured for 12 hours or more, and preferably 24 hours or more, in a suitable serum-free medium, and immunogenic substances such as serum components contained in the culture broth can be substantially removed. In addition, the culture cells can be placed in frozen storage by a conventionally known method.

A cell suspension substantially free of immunogenic substances as described above can be used as the cell composition for transplant of the present invention. This cell composition for transplant can contain cultured cells and serum-free medium.

The cell composition for transplant of the present invention can further contain (1) antibiotics, (2) nutrient components such as vitamins or glucose, (3) enzymes, (4) coenzymes, (5) antiseptics, cytokines including growth factors such as KGF or VEGF, (7) drugs such as anti-inflammatory agents, and/or (8) pigment.

In addition, the present invention relates to a method for preparing the cell composition for transplant of the present invention comprising the steps of:

(a) collecting cells from buccal fat pad;
(b) culturing the collected cells in vitro; and
(c) recovering fibroblasts from the culture.

The prepared cell composition for transplant can be administered to an affected area by a method similar to that used for cell compositions of the prior art (Japanese Unexamined International Patent Publication No. H11-510069 and Delustro, F., et al., J. Biomed. Mater. Res., 1986, 20, 109-20). For example, a cell suspension can be injected subcutaneously using a syringe. In addition, instead of being in the form of a cell suspension, the cell composition can be in the form of an agglomerate composed of fibroblasts such as a complex of fibroblasts and a matrix. Various preferable biocompatible materials, such as polysaccharides, proteins, polymeric organic materials and inorganic materials can be used for the matrix. The matrix is preferably from humans and more preferably in the form of human autologous blood or plasma, and the plasma is preferably platelet-rich plasma (PRP).

The cell composition for transplant of the present invention can preferably be administered to a subject for improving skin tissue. The subject is a mammal and preferably a human. In addition, the subject is preferably the same as the supply source of the cell composition. The improvement of skin tissue of the present invention includes the filling in of sites of deformities or defects in skin tissue in the manner of subcutaneous tissue as well as improvement of the physiological state of such sites and peripheral skin tissue thereof. Examples of defects capable of being improved by the cell composition for transplant of the present invention include wrinkles, stretch marks, depression marks, depressions in non-traumatic skin and acne vulgaris.

The dosage, number of administrations and administration interval of the cell composition for transplant for the present invention can be varied according to symptoms. A person performing the procedure such as a physician can suitably set these values according to symptoms.

Although varying according to the drug form, dosage of the active ingredient and the like, the number of cells in the cell composition is, for example, about 1×10⁵ to about 1×10⁸, preferably about 1×10⁶ to about 1×10⁸, and more preferably about 1×10⁷ to about 1×10⁸.

Although the following provides a more detailed explanation of the present invention by indicating examples thereof, these examples do not limit the scope of the present invention.

### EXAMPLE 1

### (1) Cell Culturing

### Primary Culturing, Subculturing

Fatty tissue was collected from the buccal fat pads of human adult patients in the Department of Oral Surgery of the Nagoya University Hospital (the collection of which was approved by the Medical Ethics Committee of Nagoya University and for which patient consent was obtained in advance). Collection from buccal fat pads was carried out using a syringe (18 G, Terumo Corp.), the size of the collected tissue was about 2 mm in diameter and about 1 cm in length. Abdominal adipocytes were collected in the same manner. Dermatocytes were collected by excising a 3 mm section of the skin with a scalpel.

The resulting skin tissue was treated (37° C., 2 hours) with collagenase (Wako Pure Chemical Industries, Ltd., 5 mg/ml, 2 ml per 2 mm³ of each tissue) and then washed with fresh medium to remove the collagenase. The treated skin tissue (2 mm sections) was placed in the bottom of a 6-well multiplate (diameter: 3.5 cm) and allowed to stand for 10 to 20 minutes at 25° C.

3 to 5 ml of medium (DMEM: Dulbecco's Modified Eagle's Medium, 10% serum, 1% antibiotic) were added to each well followed by culturing the cells under conditions of 37° C. and 5% CO₂. The cells grow by slowly spreading from the outer edge of the adhered tissue to the surrounding area.

After confirming growth of the cells around the tissue section, the medium was discarded, and the cells were treated with trypsin (0.05%, 5 minutes) to separate the cells from the culture dish and suspend in the medium.

The resulting cell suspension was dispensed into a culture dish containing fresh medium followed by continued culturing. In order to recover as many cells as possible, culturing was continued for all of the cells. This procedure was repeated in the case the cells had grown to confluency.

Measurement of Number of Cells (Measurement of Cell Growth Curve)

A fluorescent reagent (Cell Titer Blue, Invitrogen, 500 μl to 1 ml), for which fluorescence intensity of the culture supernatant increases corresponding to the number of viable cells, was added to the medium followed by culturing for 1 hour under conditions of 37° C. and 5% CO₂. A small amount of the culture supernatant was sampled and measured for fluorescence intensity to determine the number of viable cells from a calibration curve. The numbers of viable cells were measured over time to determine a growth curve.

In addition, the number of cells and survival rate were measured independently using a cell count analyzer (CASY Cell Counter (registered trademark), Tokyo Instruments, Inc.) after separating the cells by treating with trypsin.

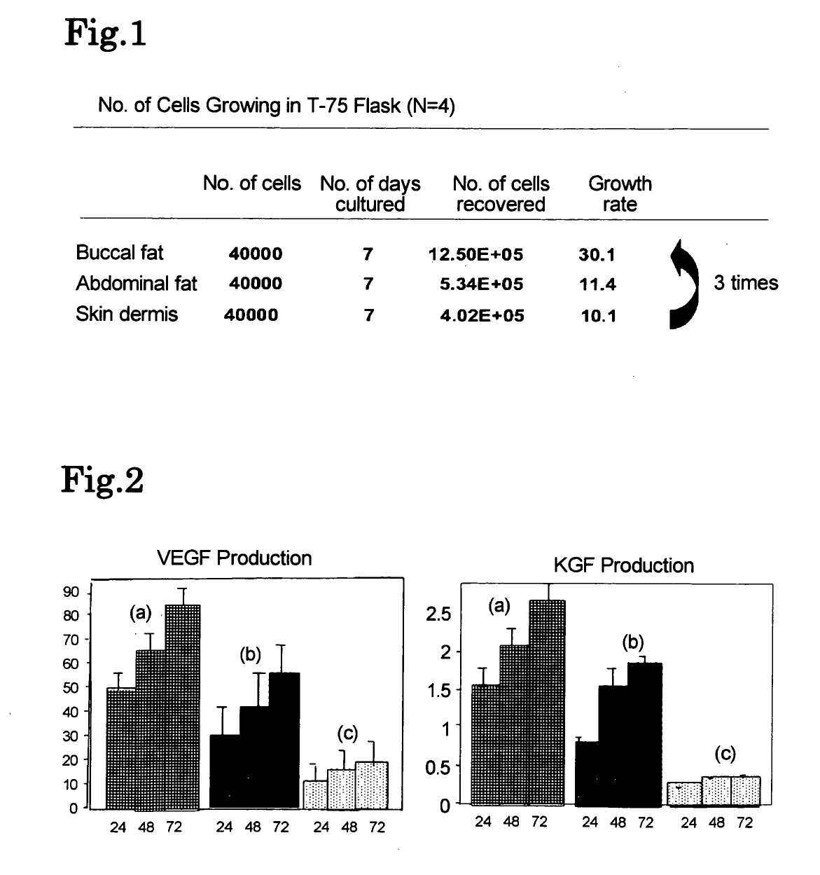

The results obtained are shown in FIG. 1. As is clear from FIG. 1, fibroblasts from buccal fat pad have a growth rate three times that of fibroblasts from skin dermis.

### EXAMPLE 2

### Production of VEGF and KGF by Buccal Fat Pad Fibroblasts

Buccal fat pad fibroblasts were seeded into each well of a 6-well plate to which had been added 2 ml of DMEM medium containing 10% fetal calf serum (Invitrogen) and 1% antibiotic-antimicotic (Invitrogen, No. 15240-062), and cultured for 1 hour under conditions of 37° C. and 5% CO₂. The medium was removed when the cells reached confluency and the cells were washed twice with PBS followed by the addition of serum-free DMEM and further culturing. 1 ml aliquots of culture broth were sampled after 24, 48 and 72 hours, and 1 ml of fresh DMEM was added to the wells. The sampled culture broth was centrifuged for 5 minutes at 4° C. and 10,000 rpm to collect the supernatant. The collected supernatant was stored at −70° C. as necessary.

The amounts of VEGF and KGF in the collected medium were measured by ELISA using a commercially available kit (n=3). The “Immunoassay Kit VEGF” (Biosource, Inc.) was used for quantitative determination of VEGF, while the “Quantikine (registered trademark) Human KGF Immunoassay Kit” (R&D Systems Co.) was used for quantitative determination of KGF.

The results are shown in FIG. 2. In both graphs, (a), (b) and (c) represent fibroblasts originating in buccal fat pad cells, abdominal adipocytes and dermatocytes moving from left to right. As is clear from FIG. 2, buccal fat pad fibroblasts have superior growth factor production capabilities as compared with other fibroblasts. The amount of VEGF produced by the buccal fat pad fibroblasts was 50 pg or more per 1 mg of supernatant protein after 24 hours of culturing in serum-free DMEM medium. In addition, the amounts of VEGF produced were confirmed to be about 65 pg or more per mg of supernatant protein after 48 hours of culturing, and about 85 pg or more after 72 hours of culturing.

The amount of KGF produced by the buccal fat pad fibroblasts was 1.5 ng or more per mg of supernatant protein after 24 hours of culturing in serum-free DMEM medium. In addition, the amounts of KGF produced were confirmed to be about 2 ng or more per mg of supernatant protein after 48 hours of culturing, and about 2.5 ng or more after 72 hours of culturing.

### EXAMPLE 3

### Administration of Cell Composition for Transplant Comprising Fibroblasts Originating in Buccal Fat Pad

Buccal fat pad fibroblasts cultured under the same conditions as in Example 1 were treated with trypsin and recovered from a culture flask. The number of recovered cells and survival rate were measured using a cell count analyzer (CASY Cell Counter (registered trademark), Tokyo Instruments, Inc.). Approximately 2×10⁷ cells were then stained using a commercially available fluorescent staining kit (PKH26 Red Fluorescent Cell Linker Mini Kit, Sigma-Aldrich Corp.).

The fluorescent stained cells were suspended in PBS and the cell density was adjusted to about 1×10⁷ cells/ml followed by injection of this cell suspension into dermis on the backs of nude mice. Tissue was collected from the injection site two months after injections, and the tissue was fixed using 4% paraformaldehyde solution (Sigma-Aldrich Corp.) for the fixative. Next, the tissue was embedded in “Tissue-Tek O.C.T. Compound (Sakura Finetechnical Co., Ltd.) followed by the preparation of about 6 μm sections.

The sections were immunostained by ELISA using anti-human collagen type I monoclonal antibody (Chemicon Corp.). Subsequently, specimens were prepared using a commercially available immunohistostaining mounting medium (Vectashield (registered trademark) Mounting Medium for Fluorescence with DAPI, Vector Laboratories, Inc.) followed by taking photomicrographs of the specimens. As a result, the transplanted buccal fat pad fibroblasts were confirmed to have remained in the dermis in the same manner as at the time of transplant even after 2 months had passed since being transplanted (or in other words, the cells were still viable). In addition, type I collagen was confirmed to be present in the cytoplasm and periphery thereof of the transplanted buccal fat pad fibroblasts. This supported the fact that buccal fat pad fibroblasts produce type I collagen even after being transplanted.

When pathological tissue at the same site was stained with hematoxylin-eosin stain and observed for regeneration of collagen fibers, active neogenesis of connective tissue was observed primarily at the cell injection site. Cellular infiltration indicative of inflammatory findings was not observed, and the new connective tissue was continuous with existing dermal components. In contrast, in a collagen injection group used as a control (5% atherocollagen, Kokensha Corp., trade name: Cellgen), the collagen was recognized as a foreign substance and active inflammatory cell infiltration was observed. This finding indicates the safety and efficacy of autologous cell transplants.

### INDUSTRIAL APPLICABILITY

The cell composition for transplant of the present invention is able to carry out repair of skin tissue and improvement of the physiological status thereof. Consequently, it can be used for skin surgical treatment including plastic surgery and cosmetic surgery.

1. A cell composition for transplant comprising fibroblasts originating in buccal fat pad.

2. The cell composition for transplant according to claim 1, wherein the buccal fat pad is of human origin.

3. The cell composition for transplant according to claim 2, wherein the buccal fat pad is autologous.

4. The cell composition for transplant according to claim 1 for improving skin tissue.

5. A method for preparing the cell composition for transplant according to claim 1, comprising the steps of:
(a) collecting cells from buccal fat pad;
(b) culturing the collected cells in vitro; and
(c) recovering fibroblasts from the culture.

6. A method for improving skin tissue comprising the step of:
(a) administering the cell composition for transplant according to claim 4 to a subject.
